SEQUENCE LISTING (2 sequences)

SEQ ID NO: 1 - protein, 13 aa, Artificial
  Synthetic peptidic inhibitor of CamKII activity
KKXLRRQEAFDAY

SEQ ID NO: 2 - protein, 13 aa, Artificial
  Synthetic peptidic inhibitor of CamKII activity
KKALHRQEAVDCL

(19) United States
(12) Patent Application Publication
(10) Pub. No.: US 2015/0329483 A1
(43) Pub. Date: Nov. 19, 2015

(54) Methods and Pharmaceutical Compositions for the Treatment of X-Linked Charcot-Marie-Tooth

(71) Applicants: INSERM (Institut National de la Santé et de la Recherche Médicale), Paris (FR); CNRS (Centre National de la Recherche Medicale), Paris (FR); Université de Strasbourg, Strasbourg (FR); Université d' Aix-Marseille, Marseille (FR); Assistance Publique-Hôpitaux de Marseille, Marseille (FR)

(72) Inventors: Michel Fontes, Marseille Cedex 5 (FR); Saleh Mones, Marseille Cedex 5 (FR); Frederic Bihel, Illkirch Cedex (FR); Claire Marsol, Illkirch Cedex (FR)

(21) Appl. No.: 14/652,934

(22) PCT Filed: Dec. 16, 2013
(86) PCT No.: PCT/EP2013/076759

§ 371 Date: Jun. 17, 2015

(30) Foreign Application Priority Data
Dec. 17, 2012 (EP) 12306594.8

(51) Int. Cl. C07C 311/21

(52) U.S. Cl. CPC C07C 311/21

(57) ABSTRACT

The present invention relates to methods and pharmaceutical compositions for the treatment of X-linked Charcot-Marie-Tooth. In particular, the present invention relates to a method for the treatment of CMTX in a subject in need thereof comprising administering the subject with a therapeutically effective amount of an inhibitor of CamKII activity or expression.

### FIELD OF THE INVENTION

The present invention relates to methods and pharmaceutical compositions for the treatment of X-linked Charcot-Marie-Tooth.

### BACKGROUND OF THE INVENTION

Charcot-Marie-Tooth disorder is a very heterogeneous inherited disorder (40 loci have been described so far, Martyn, C. N. and Hughes, 1997) affecting peripheral nerves (Dyck and Lambert, 1968). However, two forms, CMT1A and CMTX, account for at least 70% of patients with a clear familial transmission. CMT1A affects about 55% of patients and 15% suffer from CMTX (De Jonghe et al., 1999; Boerkel et al., 2002). X-linked Charcot-Marie-Tooth disease (CMTX) is an inherited X-linked peripheral neuropathy, affecting males more severely than females (Hahn et al., 1990). The average age of onset is about 16 years for males and 19 for females. It presents with slow muscular atrophy and weakness, mainly affecting the distal leg muscles. Both demyelinating and axonal anomalies are observed and clinical evaluation alone discriminates it from other CMT forms with difficulty. CMTX is caused by mutations in the gene GJB1 (Bergoffen et al., 1993), located on the proximal long arm of the X chromosome. It encodes the synthesis of connexin 32 (Cx32), a myelin membrane protein found in the PNS and CNS (Scherer et al., 1995), located in gap junctions and forming hexameric hemichannels called connexons (Mese et al., 2007). The docking of two connexons across an intercellular gap triggers the formation of a channel that connects the cytoplasms of adjacent cells (Abrams et al, 2001) and allows ions, small molecules (<1000 Da) and signalling effectors to be exchanged (Bennett et al., 1991; Liu et al., 2007). Less than 10% of the mutations observed in patients are null alleles (Abrams et al., 2001; Wang et al., 2004; Bicego et al., 2006). The majority of mutations correspond to a loss of function and could be classified into two categories: 1—Mutations affecting cell trafficking: Cx32 protein is observed in the endoplasmic reticulum and/or the Golgi but not in the cell membrane (Deschenes et al., 1997; Van Slyke et al., 2000; Yum et al., 2002). 2—Mutations affecting connexon functions: Cx32 is present in the cell membrane but the connexon cannot be opened, prohibiting any molecule exchange between adjacent cells (Omori et al., 1996; Yoshimura et al., 1996; Oh et al., 1997).

### SUMMARY OF THE INVENTION

The present invention relates to a method for the treatment of CMTX in a subject in need thereof comprising administering the subject with a therapeutically effective amount of an inhibitor of CamKII activity or expression.

### DETAILED DESCRIPTION OF THE INVENTION

X-linked Charcot-Marie-Tooth (CMTX) is the second most frequent cause of the Charcot-Marie-Tooth disorder (CMT), an inherited peripheral neuropathy. The molecular mechanism by which the locomotor system is impaired is not clearly understood and no curative treatment for patients has been proposed. The inventors have generated animal models predictive of the human CMTX phenotype, by using transgenic mice expressing a mutated human Cx32. The mutations G12S and S26L, observed in non-related CMTX families and affecting either trafficking of Cx32 (G12S) (Yum et al., 2002) or connexon activity (S26L) (Oh et al., 1997) were introduced into a human BAC containing the GJB1 gene and used to create the transgenic mouse lines. Five transgenic lines were generated, and as expected, locomotor impairments were observed Mones et al., 2012). Two lines were used in the present study: G2 (two copies of the mutated BAC, presenting a mutation affecting cell trafficking) and S3 (three copies of the mutated BAC, harbouring a mutation affecting connexon activity). All the lines present genomic instability and abnormal over-duplication of the centrosome. They also present a locomotor impairment, correlated with the number of inserted transgenes and increasing with age. The inventors demonstrate here that this is due to CamKII over-stimulation as the cellular phenotype is reverted using CamKII inhibitors (KN93 and KN62). In addition, they demonstrate that degradation of the locomotor behaviour of our transgenic lines is significantly lowered by treatment with KN93, in two lines with different point mutations in GJB1. Finally, they demonstrate that stopping the treatment leads to degradation of the phenotype. In conclusion, administration of CamKII inhibitors could be the first effective treatment for CMTX patients.

Accordingly, the present invention relates to a method for the treatment of CMTX in a subject in need thereof comprising administering the subject with a therapeutically effective amount of an inhibitor of CamKII activity or expression.

The terms “subject,” and “patient,” used interchangeably herein, refer to a mammal, particularly a human who has been previously diagnosed with CMTX or who is at risk for having or developing CMTX.

As used herein, the term “CamKII” has its general meaning in the art and refers to the Ca2+/calmodulin-dependent protein kinase II. Typically CamKII may be of any type of iso form.

An “inhibitor of CamKII activity” has its general meaning in the art, and refers to a compound (natural or not) which has the capability of reducing or suppressing the activity of CamKII (e.g. CaMKII phosphorylating activity). Typically, said inhibitor is a small organic molecule or a biological molecule (e.g. peptides, lipid, antibody, aptamer . . . ).

In a particular embodiment, the inhibitor of CamKII activity may be a small organic molecule. Example of inhibitor of CamKII activity that are small organic molecules includes, KN-93 (Sumi, M., Kiuchi, K., Ishikawa, T., Ishii, A., Hagiwara, M.1991. The newly synthesized selective Ca2+/calmodulin dependent protein kinase II inhibitor KN-93 reduces dopamine contents in PC12h cells. Biochem. Biophys. Res. Commun. 181(3):968-75), KN-62 (Tokumitsu, H., Chijiwa, T., Hagiwara, M., Mizutani, A., Terasawa, M. 1990. KN-62, 1-[N,Obis(5-isoquinolinesulfonyl)-N-methyl-L-tyrosyl]-4-phenylpiperazine, a specific inhibitor of Ca2+/calmodulin-dependent protein kinase II. J. Biol. Chem. 265 (8):4315-20), N′-{3-[1-(2-fluorobiphenyl-4-yl)-ethyl]-isoxazol5-yl}-N,N-dimethyl-guanidine hydrochloride, N-ethyl-N′-{3-[1-(2-fluorobiphenyl-4-yl)-ethyl]isoxazol-5-yl}-guanidine hydrochloride; 4-[1-(2-fluorobiphenyl-4-yl)ethyl]-2-(methylamino)-ethylthiazole or 1-[1-oxo-11-(5,6-dihydro-3-iminobenzo[h]cinnolin-2(3H)-yl)undecyl]-4-(2-pyrimidinyl)-piperazine.

In a particular embodiment, the inhibitor is selected from the group consisting of Mol CM08-16, CM08-18, CM08-33, and CM08-40 (as described in the EXAMPLE).

In another embodiment, the inhibitor of CamKII activity is a peptide. Examples of peptides include those described in US 2012/0015885 or peptides having an amino acid sequence shown as sequence Lys-Lys-X-Leu-Arg-Arg-Gln-Glu-Ala-Phe-Asp-Ala-Tyr (In the formula, X is Ala or Lys.) or a peptide having amino acid sequence shown as Lys-Lys-Ala-Leu-His-Arg-Gln-Glu-Ala-Val-Asp-Cys-Leu.

In another embodiment the inhibitor of CamKII activity is an aptamer directed against CamKII. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Peptide aptamers consists of a conformationally constrained antibody variable region displayed by a platform protein, such as E. coli Thioredoxin A that are selected from combinatorial libraries by two hybrid methods. Then after raising aptamers directed against the CamKII as above described, the skilled man in the art can easily select those blocking CamKII activity.

An “inhibitor of CamKII expression” refers to a natural or synthetic compound that has a biological effect to inhibit or significantly reduce the expression of the gene encoding for CamKII.

Inhibitors of expression for use in the present invention may be based on anti-sense oligonucleotide constructs. Anti-sense oligonucleotides, including anti-sense RNA molecules and anti-sense DNA molecules, would act to directly block the translation of CamKII mRNA by binding thereto and thus preventing protein translation or increasing mRNA degradation, thus decreasing the level of CamKII, and thus activity, in a cell. For example, antisense oligonucleotides of at least about 15 bases and complementary to unique regions of the mRNA transcript sequence encoding CamKII can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of expression for use in the present invention. CamKII gene expression can be reduced by contacting a subject or cell with a small double stranded RNA (d5RNA), or a vector or construct causing the production of a small double stranded RNA, such that CamKII gene expression is specifically inhibited (i.e. RNA interference or RNAi). Methods for selecting an appropriate dsRNA or dsRNA-encoding vector are well known in the art for genes whose sequence is known. All or part of the phosphodiester bonds of the siRNAs of the invention are advantageously protected. This protection is generally implemented via the chemical route using methods that are known by art. The phosphodiester bonds can be protected, for example, by a thiol or amine functional group or by a phenyl group. The 5′- and/or 3′-ends of the siRNAs of the invention are also advantageously protected, for example, using the technique described above for protecting the phosphodiester bonds. The siRNAs sequences advantageously comprises at least twelve contiguous dinucleotides or their derivatives.

As used herein, the term “siRNA derivatives” with respect to the present nucleic acid sequences refers to a nucleic acid having a percentage of identity of at least 90% with erythropoietin or fragment thereof, preferably of at least 95%, as an example of at least 98%, and more preferably of at least 98%.

As used herein, “percentage of identity” between two nucleic acid sequences, means the percentage of identical nucleic acid, between the two sequences to be compared, obtained with the best alignment of said sequences, this percentage being purely statistical and the differences between these two sequences being randomly spread over the nucleic acid acids sequences. As used herein, “best alignment” or “optimal alignment”, means the alignment for which the determined percentage of identity (see below) is the highest. Sequences comparison between two nucleic acids sequences are usually realized by comparing these sequences that have been previously align according to the best alignment; this comparison is realized on segments of comparison in order to identify and compared the local regions of similarity. The best sequences alignment to perform comparison can be realized, beside by a manual way, by using the global homology algorithm developed by SMITH and WATERMAN (Ad. App. Math., vol. 2, p: 482, 1981), by using the local homology algorithm developed by NEDDLEMAN and WUNSCH (J. Mol. Biol., vol. 48, p: 443, 1970), by using the method of similarities developed by PEARSON and LIPMAN (Proc. Natl. Acd. Sci. USA, vol. 85, p: 2444, 1988), by using computer software using such algorithms (GAP, BESTFIT, BLAST P, BLAST N, FASTA, TFASTA in the Wisconsin Genetics software Package, Genetics Computer Group, 575 Science Dr., Madison, Wis. USA), by using the MUSCLE multiple alignment algorithms (Edgar, Robert C., Nucleic Acids Research, vol. 32, p: 1792, 2004). To get the best local alignment, one can preferably used BLAST software. The identity percentage between two sequences of nucleic acids is determined by comparing these two sequences optimally aligned, the nucleic acids sequences being able to comprise additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences.

shRNAs (short hairpin RNA) can also function as inhibitors of expression for use in the present invention.

Ribozymes can also function as inhibitors of expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of CamKII mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable.

Both antisense oligonucleotides and ribozymes useful as inhibitors of expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and preferably cells expressing CamKII. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adenoviruses and adeno-associated (AAV) viruses, which are double-stranded DNA viruses that have already been approved for human use in gene therapy. Actually 12 different AAV serotypes (AAV1 to 12) are known, each with different tissue tropisms (Wu, Z Mol Ther 2006; 14:316-27). Recombinant AAV are derived from the dependent parvovirus AAV2 (Choi, V W J Virol 2005; 79:6801-07). The adeno-associated virus type 1 to 12 can be engineered to be replication deficient and is capable of infecting a wide range of cell types and species (Wu, Z Mol Ther 2006; 14:316-27). It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the antisense oligonucleotide, siRNA, shRNA or ribozyme nucleic acid sequence is under the control of a heterologous regulatory region, e.g., a heterologous promoter.

The inhibitor of CamKII activity or expression may be combined with pharmaceutically acceptable excipients, and optionally sustained-release matrices, such as biodegradable polymers, to form therapeutic compositions.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The inhibitor of CamKII activity or expression of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The inhibitor of CamKII activity or expression of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

The present invention also relates to a method for screening a plurality of candidate compounds for use as a drugs for the prevention and treatment of CMTX comprising the steps consisting of (a) testing each of the candidate compounds for its ability to inhibit CamKII activity or expression and (b) and positively selecting the candidate compounds capable of inhibiting said CamKII activity or expression.

Typically, the candidate compound is selected from the group consisting of small organic molecules, peptides, polypeptides or oligonucleotides. Other potential candidate compounds include antisense molecules, siRNAs, or ribozymes. Testing whether a candidate compound can inhibit CamKII activity or expression can be determined using or routinely modifying assays known in the art. For example, methods for assaying the inhibitory effects of a compound on CamKII activity are well known in the art. Typically such a method is described in Sumi, M., Kiuchi, K., Ishikawa, T., Ishii, A., Hagiwara, M.1991. The newly synthesized selective Ca2+/calmodulin dependent protein kinase II inhibitor KN-93 reduces dopamine contents in PC12h cells. Biochem. Biophys. Res. Commun. 181(3):968-75. The candidate compounds that have been positively selected may be subjected to further selection steps in view of further assaying its properties for a development int the therapy of CMTX. For example, the candidate compounds that have been positively selected may be subjected to further selection steps in view of further assaying their in vitro or in vivo properties. Typically in vitro assays include but are not limited to assays for determining whether the compounds are able to rescue the connexion activity (such as described in EXAMPLE) and in vivo assays typically include assays on animal models for CMTX. Typically, such animal models include those described in EXAMPLE, namely transgenic mice expressing a mutated connexion 32 such as G2 or S3 lines. Then the locomotor impairment may be evaluated as described in the EXAMPLE.

The invention will be further illustrated by the following figures and examples. However, these examples and figures should not be interpreted in any way as limiting the scope of the present invention.

### FIGURES

FIG. 1. Fibroblasts from wild type mice (WT) or transgenic lines (G2 or S3) were collected and cultured. A. Protein extract was obtained from WT and S3 fibroblasts, western blotted and probed with an antibody against phosphorylated CamKII. B. Proteins from fibroblasts from either WT animals or S3 transgenic animals have been analyzed on an acrylamide gel and blotted. Membrane have been the probed with an antibody directed toward the phosphorylated form of CamKII.

FIG. 2. A. Centrosomes of cultured WT fibroblasts were stained with an antibody raised against γ-tubulin (in red). B. Centrosomes of cultured S3 transgenic fibroblasts were stained with an antibody raised against γ-tubulin (in red). An abnormal number of centrosomes could be observed. C. Nuclear volumes of S3 fibroblasts and S3 fibroblasts treated either with KN93 or KN62 were evaluated after DAPI staining, using ImageJ software. D. Fibroblasts from WT, S3 and S3 treated with KN93 were cultured and the percentages of cells with one centrosome (white), two centrosomes (red), and more than two (blue) were determined. This analysis has been repeated three times.

FIG. 3. A. Connexon activity in fibroblasts from WT, S3 and S3 treated with KN93 was monitored in a 96 well plate assay, using lucifer yellow as a fluorescent dye (see methods). Fluorescence is in arbitrary units. B, C and D. Connexon activity of WT mouse fibroblasts (B), S3 transgenic mouse (C) and S3 incubated with KN93 (D), were incubated with LY for two hours and examined under a fluorescent microscope.

FIG. 4. Mice from the G2 line were treated either with a placebo (4 animals) or with soluble KN93 (4 animals, 1.5 mg/kg, i.p., 1 administration per day) for one month and performances on the rotarod evaluated. Treatment was then stopped and locomotor performances evaluated one month later.

### EXAMPLE 1

### Synthesis of KN93 Derivatives: General Procedures

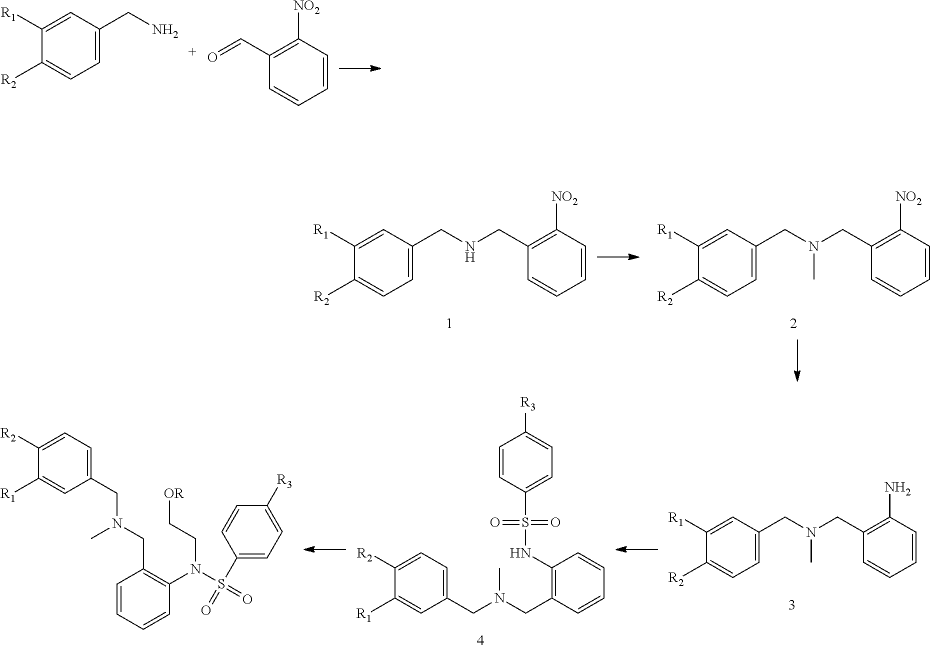

Reductive Amination Using NaBH₄ (1)

2-Nitrobenzaldehyde (1 equiv) was added to a stirring solution of benzylamine (1 equiv) in MeOH and stirred at 50° C. for 6 hours. The reaction mixture was cooled to 0° C. and NaBH₄ (2 equiv) was added slowly. The reaction mixture was allowed to reach room temperature and was left stirring overnight. NaOH (2M) was added and the crude product was extracted using EtOAc. The organic layers were combined, washed with brine, dried over Na₂SO₄, filtered and concentrated under reduced pressure. The crude product was purified by flash chromatography (EtOAc/heptane 1/1)

N-Methylation (2)

To a stirring solution of secondary amine (1 equiv) in CH₂Cl₂ was added aq. Formaldehyde (37% solution) (1.1 equiv) and formic acid (2.5 equiv) and the reaction mixture was heated to reflux overnight. The reaction mixture was partitioned between CH₂Cl₂ and a saturated aqueous solution of K₂CO₃. The layers were washed with brine, dried over Na₂SO₄, and concentrated under reduced pressure. The crude product was purified using flash chromatography (EtOAc/heptane 1/1).

General Procedure: Reduction of the Nitro Group Using SnCl₂ (3)

SnCl₂ (5 equiv) was added to a stirring solution of the nitro compound (1 equiv) in methanol. The reaction mixture was heated to reflux for 2 hours and then allowed to cool to room temperature. The pH was adjusted to 8 by addition of a saturated aqueous solution of NaHCO₃. EtOAc was added, the layers were separated and the aqueous layer was extracted with EtOAc. The combined organic layers were washed with brine, dried over Na₂SO₄, and concentrated under reduced pressure. The crude product was purified using flash chromatography (EtOAc/heptane 1/1).

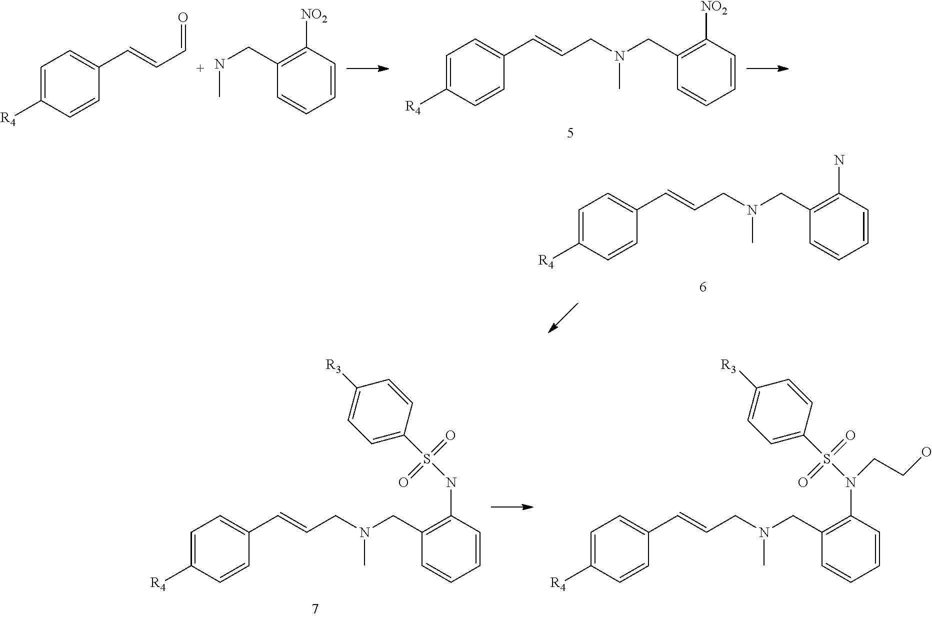

The same protocol was used starting from 3-(4-chlorophenyl)propanal.

General Procedure: Reaction Between Aldehyde and N-methyl-1-(2-nitrophenyl)methanamine (5)

3-(4-chlorophenyl)propanal was obtained by reduction of the corresponding acid in alcohol, followed by Swern oxidation.

A solution of aldehyde (1 equiv), N-methyl-1-(2-nitrophenyl)methanamine (2.8 equiv) and NaBH₃CN (0.7 equiv) in MeOH was stirred for 15 min at 110° C. in a microwave reactor. HCl 6N was added to destroy excess of NaBH₃CN. The aqueous solution was made alkaline and extracted with Et₂O. The organic layer was dried over Na₂SO₄, and concentrated under reduced pressure. The crude product was purified using flash chromatography (EtOAc/heptane 1/9).

General Procedure: Reduction of the Nitro Group Using Zn Dust (6)

A solution of NH₄Cl (2.4 equiv) in H₂O (0.2 mL/mmol) was added to a stirring solution of the nitro compound (1 equiv) in acetone. The mixture was brought to reflux and Zn dust (5 equiv) was added in small portions. After 6 hours, the mixture was filtered and the solid washed with CH₂Cl₂. The organic layer was dried over Na₂SO₄, and concentrated under reduced pressure. The crude product was purified using flash chromatography (EtOAc/heptane 3/7).

General Procedure: Introduction of the Sulfonamide Group (4 and 7)

To a stirring solution of the amine (1 equiv) in CH₂Cl₂ at 0° C. was added pyridine (3 equiv), and then a solution of the sulfonyl chloride (1.2 equiv) in CH₂Cl₂. The reaction mixture was stirred at room temperature for 3 hours. The reaction mixture was concentrated and the residue was taken up with water, made alkaline with NaHCO₃ and extracted with CH₂Cl₂. The combined organic layers were washed with brine, dried over Na₂SO₄, and concentrated under reduced pressure. The crude product was purified using flash chromatography (EtOAc/heptane 2/8).

General Procedure: Reaction with Ethylene Carbonate (or 1-bromo-2-methoxyethane for CM07-118)

To a stirring solution of the amino sulfonamide compound (1 equiv) in DMF was added NaOH (1 equiv) and ethylene carbonate (or 1-bromo-2-methoxyethane for CM07-118) (4.5 equiv). The reaction mixture was refluxed overnight. The reaction mixture was concentrated and the residue was partitioned between EtOAc and brine. The organic layer was dried over Na₂SO₄, and concentrated under reduced pressure. The crude product was purified using flash chromatography (EtOAc/heptane 1/1 until EtOAc 100%).

### EXAMPLE 2

### N-[2-({[(2E)-3-(4-chlorophenyl)prop-2-en-1-yl](methyl)amino}methyl)phenyl]-2-hydroxy-S′-phenylethane-1-sulfonamido (CM07-109)

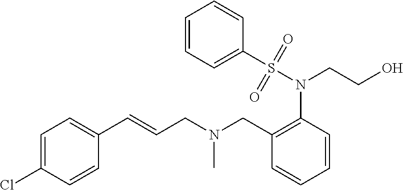 [SMILES: CN(C/C=C/c1ccc(Cl)cc1)Cc1ccccc1N(CCO)S(=O)(=O)c1ccccc1]

¹H NMR (300 MHz, CDCl₃) δ=2.22 (s, 3H), 2.90 (dd, J=9 Hz, J=3 Hz, 1H), 3.10 (dd, J=9 Hz, J=3 Hz, 1H), 3.26-3.43 (m, 2H), 3.65-3.69 (m, 1H), 4.12 (td, J=9 Hz, J=3 Hz, 1H), 4.99 (m, 1H), 6.37-6.52 (m, 3H), 7.14-7.20 (m, 1H), 7.25-7.34 (m, 4H), 7.39-7.42 (m, 2H), 7.47-7.53 (m, 2H), 7.60-7.63 (m, 1H). ¹³C NMR (400 MHz, CDCl₃) δ=133.6, 133.0, 130.2, 128.8, 128.7, 128.6, 128.5, 128.1, 127.9, 127.3, 126.0, 60.4, 59.1, 58.8, 55.8, 41.5. MS (ESI+) M/Z found (M+H)⁺ 471.2.

### EXAMPLE 3

### N-[2-({[(2E)-3-(4-chlorophenyl)prop-2-en-1-yl](methyl)amino}methyl)phenyl]-4-methoxy-N-(2-methoxyethyl)benzene-1-sulfonamide (CM07-118)

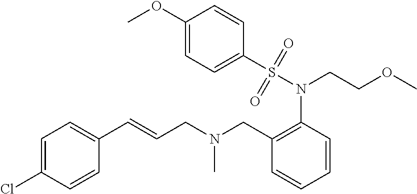 [SMILES: COCCN(c1ccccc1CN(C)C/C=C/c1ccc(Cl)cc1)S(=O)(=O)c1ccc(OC)cc1]

MS (ESI+) M/Z found (M+H)⁺ 515.2.

### EXAMPLE 4

### 2-hydroxy-S-(4-methoxyphenyl)-N′-[2-({methyl[(2E)-3-phenylprop-2-en-1-yl]amino}methyl)phenyl]ethane-1-sulfonamido (CM08-16)

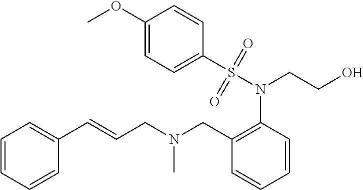 [SMILES: COc1ccc(S(=O)(=O)N(CCO)c2ccccc2CN(C)C/C=C/c2ccccc2)cc1]

¹H NMR (400 MHz, CDCl₃) δ=2.15 (s, 3H), 2.85 (d, J=12 Hz, 1H), 3.01 (dd, J=12 Hz, J=4 Hz, 1H), 3.17-3.22 (m, 2H), 3.30-3.33 (m, 1H), 3.60 (dt, J=12 Hz, J=4 Hz, 1H), 3.79 (s, 3H), 3.95-4.04 (m, 1H), 4.88 (d, J=16 Hz, 1H), 6.30-6.39 (m, 2H), 6.50 (d, J=16 Hz, 1H), 6.85-6.86 (m, 2H), 7.08-7.24 (m, 7H), 7.36-7.38 (m, 2H), 7.45-7.48 (m, 2H). ¹³C NMR (400 MHz, CDCl₃) δ=134.2, 133.5, 130.6, 128.7, 128.5, 128.0, 127.6, 127.4, 126.7, 125.2, 113.9, 60.5, 59.3, 59.1, 55.7, 55.6, 41.5. MS (ESI+) M/Z found (M+H)⁺ 467.2.

### EXAMPLE 5

### 2-hydroxy-S-(4-methoxyphenyl)-N′-(2-{[methyl(naphthalen-2-ylmethyl)amino]methyl}phenyl)ethane-1-sulfonamido (CM08-18)

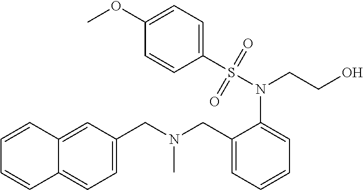 [SMILES: COc1ccc(S(=O)(=O)N(CCO)c2ccccc2CN(C)Cc2ccc3ccccc3c2)cc1]

¹H NMR (400 MHz, CDCl₃) δ=2.07 (s, 3H), 2.90 (d, J=12 Hz, 1H), 3.03 (dt, J=16 Hz, J=4 Hz, 1H), 3.24 (td, J=16 Hz, J=4 Hz, 1H), 3.61 (dt, J=16 Hz, J=4 Hz, 1H), 3.76 (d, J=12 Hz, 1H), 3.78 (s, 3H), 3.91 (d, J=12 Hz, 1H), 4.02 (td, J=16 Hz, J=4 Hz, 1H), 4.90 (d, J=12 Hz, 1H), 6.39 (d, J=8 Hz, 1H), 6.81-6.84 (m, 2H), 7.06-7.23 (m, 3H), 7.37-7.50 (m, 5H), 7.62-7.79 (m, 4H). ¹³C NMR (400 MHz, CDCl₃) δ=133.6, 130.6, 129.2, 128.6, 128.1, 128.0, 127.9, 127.7, 127.4, 126.0, 125.9, 113.9, 63.3, 59.4, 59.3, 55.65, 55.62, 41.0. MS (ESI+) M/Z found (M+H)⁺ 491.2.

### EXAMPLE 6

### N-(2-{[benzyl(methyl)amino]methyl}phenyl)-2-hydroxy-S′-(4-methoxyphenyl)ethane-1-sulfonamido (CM08-33)

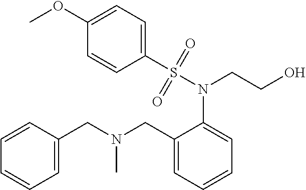 [SMILES: COc1ccc(S(=O)(=O)N(CCO)c2ccccc2CN(C)Cc2ccccc2)cc1]

¹H NMR (400 MHz, CDCl₃) δ=2.02 (s, 3H), 2.82 (d, J=12 Hz, 1H), 2.99-3.02 (m, 1H), 3.14-3.19 (m, 1H), 3.51-3.54 (m, 2H), 3.71-3.78 (m, 1H), 3.78 (s, 3H), 9.97-4.00 (m, 1H), 4.75-4.78 (m, 1H), 6.36 (d, J=8 Hz, 1H), 6.86-6.88 (m, 2H), 7.09 (m, 1H), 7.18-7.31 (m, 7H), 7.41-7.43 (m, 2H). ¹³C NMR (400 MHz, CDCl₃) δ=133.6, 130.4, 130.2, 128.7, 128.3, 128.1, 127.6, 127.3, 114.0, 64.8, 63.1, 59.0, 55.6, 55.5, 41.1. MS (ESI+) M/Z found (M+H)⁺ 441.2.

### EXAMPLE 7

### N-[2-({[3-(4-chlorophenyl)propyl](methyl)amino}methyl)phenyl]-2-hydroxy-S′-(4-methoxyphenyl)ethane-1-sulfonamido (CM08-40)

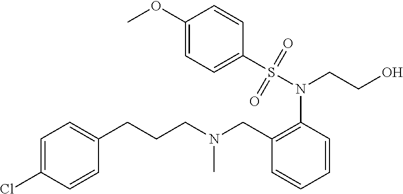 [SMILES: COc1ccc(S(=O)(=O)N(CCO)c2ccccc2CN(C)CCCc2ccc(Cl)cc2)cc1]

¹H NMR (400 MHz, CDCl₃) δ=1.91-1.97 (m, 2H), 2.09 (s, 3H), 2.34-2.41 (td, J=12 Hz, J=4 Hz, 1H), 2.53-259 (m, 3H), 2.73 (d, J=12 Hz, 1H), 2.98 (dd, J=12 Hz, J=4 Hz, 1H), 3.13 (td, J=12 Hz, J=4 Hz, 1H), 3.55 (dt, J=12 Hz, J=4 Hz, 1H), 3.80 (s, 3H), 3.97 (td, J=12 Hz, J=4 Hz, 1H), 3.85 (d, J=12 Hz, 1H), 6.35 (d, J=8 Hz, 1H), 6.86-6.89 (m, 2H), 7.07-7.18 (m, 7H), 7.45-7.48 (m, 2H). ¹³C NMR (400 MHz, CDCl₃) δ=133.6, 130.6, 129.9, 128.6, 128.4, 128.0, 127.3, 113.9, 59.2, 59.1, 57.9, 55.7, 55.6, 41.9, 33.2, 27.4. MS (ESI+) M/Z found (M+H)⁺ 503.2.

### EXAMPLE 8

### Synthesis of KN62 Derivatives: General Procedures

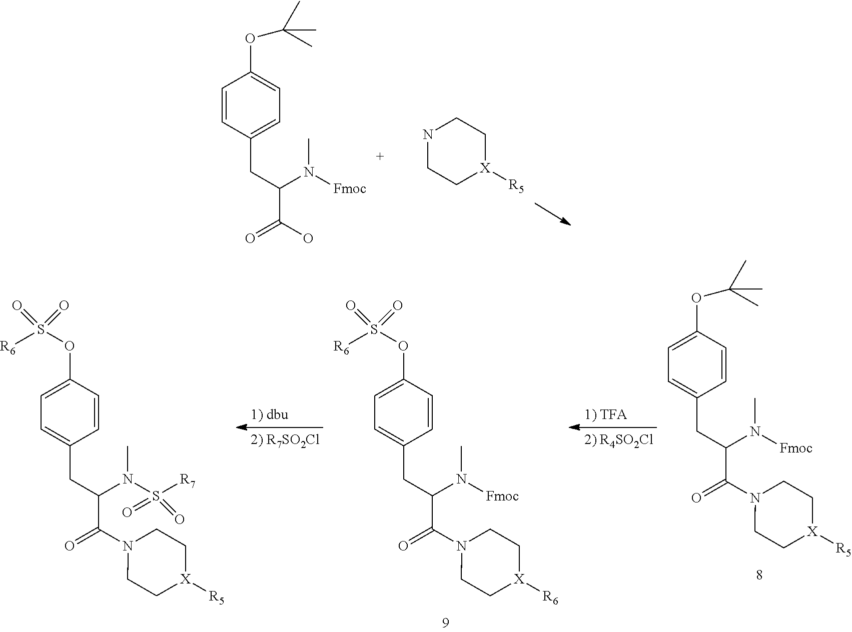

General Procedure: Reaction Between Tyrosine and Amine (8)

To a stirring solution of fmoc-tyrosine(tBu)-OH (1 equiv) in DMF at 0° C., was added EDC (1.1 equiv), HOBt (1.1 equiv) and the amine (1.1 equiv). The reaction mixture was stirred at room temperature for 24 hours. DMF was evaporated and the residue dissolved in EtOAc, washed with water and brine. The organic layer was dried over Na₂SO₄, and concentrated under reduced pressure. The crude product was purified using flash chromatography (EtOAc/heptane 1/1).

For CM07-161, the coupling reaction was done using BOP (1.05 equiv)/DIEA (3 equiv) conditions.

General Procedure: Deprotection of the tert-butyloxyphenol

To a stirring solution of the tert-butyloxyphenol derivatives (1 equiv) in CH₂Cl₂, was added TFA (20 equiv). The reaction mixture was concentrated and the residue was taken up with water, made alkaline with NaHCO₃ and extracted with CH₂Cl₂. The combined organic layers were dried over Na₂SO₄, and concentrated under reduced pressure. The residue obtained was used for the next reaction without any purification.

General Procedure: Sulfonylation of the Phenol (9)

To a suspension of NaH (1.2 equiv) in dry THF was added phenol derivatives (1 equiv). After 10 min, sulfonyl chloride (2 equiv) in CH₂Cl₂ was added. The reaction mixture was stirred 18 hours at room temperature and concentrated. The residue was dissolved in EtOAc and washed with NaHCO₃. The organic layer was dried over Na₂SO₄, and concentrated under reduced pressure. The crude product was purified using flash chromatography (EtOAc/heptane 1/1).

General Procedure: Fmoc Deprotection

To a stirring solution of the Fmoc compound (1 equiv) in CH₂Cl₂ was added dbu (2 equiv). The reaction mixture was stirred at room temperature for 18 hours. After evaporation under reduced pressure, the crude product was purified using flash chromatography (EtOAc/MeOH 9/1).

General Procedure: Sulfonylation of the Amine

To a stirring solution of the amine (1 equiv) in CH₂Cl₂, was added Et₃N (1.5 equiv) and the sulfonyl chloride (2 equiv). The reaction mixture was stirred at room temperature for 18 hours. The reaction mixture was washed with NaHCO₃, water and brine. The organic layer was dried over Na₂SO₄, and concentrated under reduced pressure. The crude product was purified using flash chromatography (CH₂Cl₂/MeOH 9/1).

For CM07-140, deprotection of the tert-butyloxyphenol was done in the last step after sulfonylation of the amine.

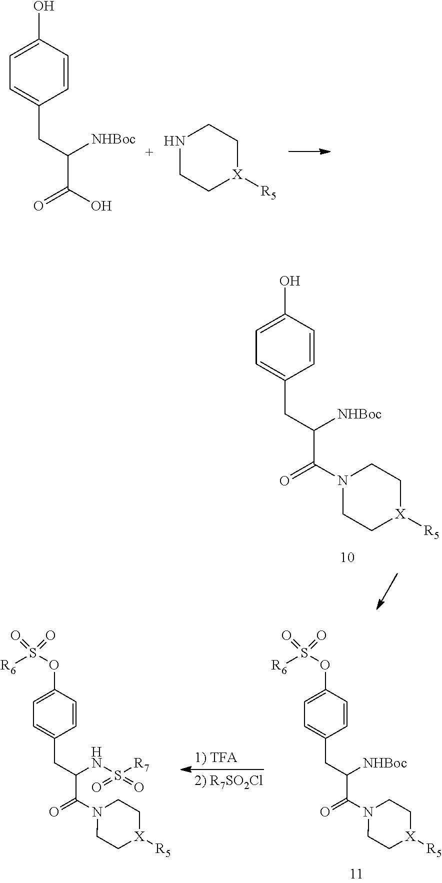

For CM07-132, the synthesis was done by reaction between N-Boc-tyrosine and phenyl piperazine (10) following general procedure (8), then the sulfonylation of the phenol (11) was also done following general procedure. The removal of the Boc protecting group was done using standards conditions (TFA/CH₂Cl₂). The product was then obtained by sulfonylation of the amine following general procedure.

### EXAMPLE 7

### isoquinoline-5-sulfonamido)-3-oxo-3-(4-phenylpiperazin-1-yl)propyl]phenyl isoquinoline-5-sulfonate (CM07-132)

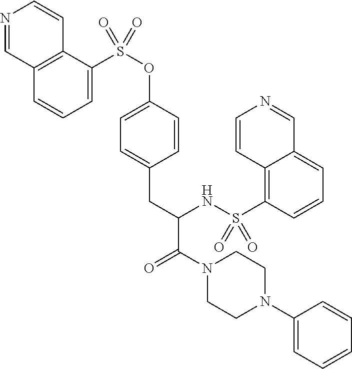 [SMILES: O=C(C(Cc1ccc(OS(=O)(=O)c2cccc3cnccc23)cc1)NS(=O)(=O)c1cccc2cnccc12)N1CCN(c2ccccc2)CC1]

¹H NMR (400 MHz, CDCl₃) δ=2.57-2.64 (m, 2H), 2.69-2.89 (m, 5H), 2.99-3.14 (m, 2H), 3.23-3.31 (m, 1H), 4.29-4.37 (m, 1H), 6.14-6.19 (bd, 1H, J=9 Hz), 6.63-6.67 (m, 2H), 6.79-6.82 (d, 2H, J=7 Hz), 6.87-6.96 (m, 2H), 7.48-7.53 (t, 1H, J=6 Hz), 7.57-7.62 (t, 1H, J=6 Hz), 8.13-8.16 (td, 2H, J=7 Hz, J=2 Hz), 8.21-8.24 (d, 1H, J=9 Hz), 8.28-8.32 (dt, 2H), J=7 Hz, J=1 Hz), 8.50 (d, 1H, J=6 Hz), 8.68 (d, 1H, J=6 Hz), 8.81 (d, 1H, J=6 Hz), 9.28 (s, 1H), 9.39 (s, 1H). ¹³C NMR (400 MHz, CDCl₃) δ=153.3, 153.2, 145.9, 145.4, 135.1, 134.8, 133.8, 132.5, 130.7, 129.3, 125.7, 125.6, 122.0, 121.0, 117.5, 117.1, 116.6, 53.5, 53.4, 48.9, 48.7, 44.9, 41.7, 39.9. MS (ESI+) M/Z found (M+H)¹ 708.2

### EXAMPLE 8

### 2-(N-methylbenzenesulfonamido)-3-oxo-3-(4-phenylpiperazin-1-yl)propyl]phenyl isoquinoline-5-sulfonate (CM07-136)

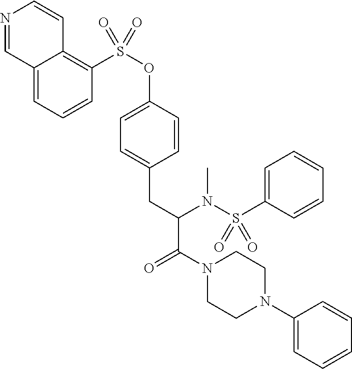 [SMILES: CN(C(Cc1ccc(OS(=O)(=O)c2cccc3cnccc23)cc1)C(=O)N1CCN(c2ccccc2)CC1)S(=O)(=O)c1ccccc1]

¹H NMR (400 MHz, CDCl₃) δ=2.27-2.32 (dd, 1H J=12 Hz, J=3 Hz), 2.75-2.82 (m, 1H), 2.92 (s, 3H), 2.98-3.08 (m, 5H), 3.34-3.53 (m, 2H), 3.64-3.79 (m, 2H), 4.98-5.03 (dd, 1H, J=10 Hz, J=4 Hz), 6.76-6.79 (m, 2H), 6.88-6.94 (m, 3H), 6.98-7.01 (m, 2H), 7.27-7.32 (m, 2H), 7.48-7.61 (m, 4H), 7.75-7.78 (m, 2H), 8.18 (dd, 1H, J=7 Hz, J=1 Hz), 8.23 (d, 1H, J=8 Hz), 8.51 (d, 1H, J=6 Hz), 8.79 (d, 1H, J=6 Hz), 9.40 (s, 1H). ¹³C NMR (400 MHz, CDCl₃) δ=0.153.2, 145.8, 135.0, 134.7, 133.0, 130.6, 129.3, 127.2, 122.1, 117.4, 116.5, 56.0, 49.2, 45.2, 41.9, 33.6, 30.5, 30.4 MS (ESI+) M/Z found (M+H)⁺ 671.2

### EXAMPLE 9

### 3-(4-hydroxyphenyl)-S-(isoquinolin-5-yl)-N-methyl-1-oxo-1-(4-phenylpiperazin-1-yl)propane-2-sulfonamido (CM07-140)

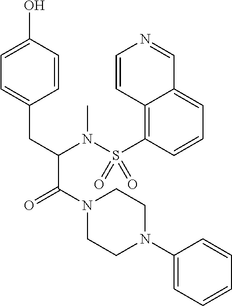 [SMILES: CN(C(Cc1ccc(O)cc1)C(=O)N1CCN(c2ccccc2)CC1)S(=O)(=O)c1cccc2cnccc12]

¹H NMR (300 MHz, CDCl₃) δ=0.2.56-2.63 (dd, 1H, J=13 Hz, J=6 Hz), 2.67-2.76 (m, 1H), 2.99-3.13 (m, 4H), 3.18 (s, 3H), 3.43-3.51 (m, 1H), 3.63-3.73 (m, 3H), 5.08-5.13 (dd, 1H, J=8 Hz, J=6), Hz), 6.59-6.62 (d, 2H, J=5 Hz), 6.83-6.95 (m, 5H), 7.24-7.29 (m, 3H), 7.69-7.74 (t, 1H, J=15 Hz), 8.23 (d, 1H, J=8 Hz), 8.35-8.39 (dd, 2H, J=8 Hz, J=6 Hz), 8.62 (d, 1H, J=6 Hz), 9.35 (s, 1H). ¹³C NMR (400 MHz, CDCl₃) δ=152.8, 144.3, 133.8, 133.4, 130.2, 129.2, 126.0, 116.6, 115.5, 54.8, 54.7, 49.3, 45.9, 41.8, 35.1, 30.9. MS (ESI+) M/Z found (M+H)⁺ 531.2

### EXAMPLE 10

### 4-[2-(N-methylisoquinoline-5-sulfonamido)-3-oxo-3-(4-phenylpiperazin-1-yl)propyl]phenyl benzenesulfonate (CM07-143)

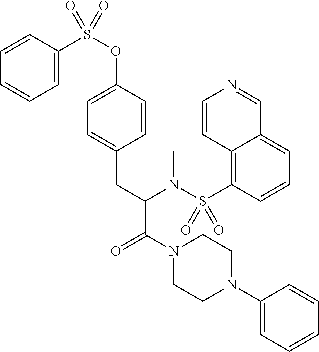 [SMILES: CN(C(Cc1ccc(OS(=O)(=O)c2ccccc2)cc1)C(=O)N1CCN(c2ccccc2)CC1)S(=O)(=O)c1cccc2cnccc12]

¹H NMR (300 MHz, CDCl₃) δ=2.48-2.54 (dd, 1H, J=13 Hz, J=4 Hz), 2.65-2.75 (m, 1H), 2.92-3.05 (m, 4H), 3.08 s, 3H), 3.21-3.39 (m, 2H), 3.52-3.67 (m, 2H), 5.14-5.19 (dd, J=10 Hz, J=4 Hz), 6.83-6.92 (m, 4H), 7.02-7.05 (d, 2H, J=9 Hz), 7.24-7.29 (m, 2H), 7.44-7.49 (t, 2H, J=8 Hz), 7.616 7.66 (t, 1H, J=7 Hz), 7.67-7.74 (t, 1H, J=8 Hz), 7.77-7.81 (m, 2H), 8.23 (d, 1H, J=8 Hz), 8.32 (d, 1H, J=8 Hz), 8.45 (d, 1H, J=8 Hz), 8.69 (d, 1H, J=8 Hz), 9.36 (s, 1H). ¹³C NMR (400 MHz, CDCl₃) δ=153.4, 145.2, 134.2, 134.0, 133.0, 130.4, 129.3, 129.1, 128.4, 125.8, 122.6, 120.6, 117.4, 116.6, 54.9, 49.3, 445.5, 41.8, 35.1, 30.7, 29.7. MS (ESI+) M/Z found (M+H)⁺

### EXAMPLE 11

### 4-[2-(N-methylisoquinoline-5-sulfonamido)-3-oxo-3-(piperidin-1-yl)propyl]phenyl isoquinoline-5-sulfonate (CM07-161)

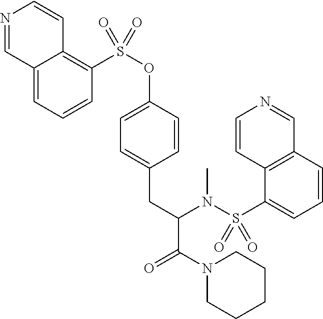 [SMILES: CN(C(Cc1ccc(OS(=O)(=O)c2cccc3cnccc23)cc1)C(=O)N1CCCCC1)S(=O)(=O)c1cccc2cnccc12]

¹H NMR (400 MHz, CDCl₃) δ=1.26-1.41 (m, 6H), 2.36-2.41 (dd, 1H, J=13 Hz, J=4 Hz), 2.95 (s, 3H), 3.09-3.15 (4H), 3.37-3.43 (m, 1H), 4.99-5.03 (dd, 1H, J=10 Hz, J=4 Hz), 6.67-6.69 (dd, 2H, J=7 Hz, J=2 Hz), 6.88-6.90 (dd, 2H, J=7 Hz, J=2 Hz), 7.54-7.62 (m, 2H), 8.12 (d, 1H, J=8 Hz), 8.16-8.23 (m, 3H), 8.33 (d, 1H, J=6 Hz), 8.44 (d, 1H, J=6 Hz), 8.72 (d, 1H, J=6 Hz), 9.25 (s, 1H), 9.34 (s, 1H). ¹³C NMR (400 MHz, CDCl₃) δ=153.4, 153.3, 145.9, 145.1, 135.2, 134.7, 133.8, 132.9, 130.6, 125.8, 125.7, 122.1, 117.5, 117.4, 54.8, 46.9, 43.2, 35.2, 30.8, 26.3, 25.6, 24.2. MS (ESI+) M/Z found (M+H)⁺ 645.0

### EXAMPLE 13

### Effects of KN93 and KN62

### Material & Methods

Generation of Transgenic Lines

BAC modifications were generated by Gene Bridges GmbH Heidelberg using recombineering technology. BAC DNA was isolated from preparative pulsed field gels using a modification of a previously described method (Huxley et al., 1996). Transgenic mice were generated using the standard technique of pronuclear injection using C57BL/6J—CBA/Ca F1 mice as donors. Subsequent crosses were to the same F1 mice.

Cell Culture

For the isolation of fibroblasts a small fragment of mouse ear was removed, dipped in alcohol solution, cut into small pieces in a sterile Petri dish in the presence of PBS containing fungicide (fonigizon) diluted 1/250, and transferred to 2-ml tubes containing 1 ml dissociation buffer (DMEM plus 20% FBS, 1 mg/ml BSA, 0.5 mg/ml collagenase, 0.25 mg/ml trypsin and penicillin/streptomycin). The tubes were incubated in a water bath with agitation for 1 h at 37° C. Fibroblast medium (DMEM, 10% FBS, 2 mM Gln, 100 U/ml penicillin, 100 μg/ml streptomycin) was added to each tube and the samples were centrifuged at 400 g for 10 min. The cells were re-suspended in fibroblast medium, seeded into Petri dishes and placed in an incubator at 37° C., 5% CO2. The culture medium was replaced every two days. When the cultures reached sub-confluence, the cells were trypsinized and expanded into tissue culture flasks. All experiments were performed using cells between passage numbers four and eight.

Western Blotting

Cells were lysed in RIPA buffer (50 mM Tris-Cl pH 7.4, 1% NP40, 0.25% sodium deoxycholate, 0.1% SDS, 150 mM sodium chloride) supplemented with protease and phosphatase inhibitors. The same amounts of protein from each sample were resolved under denaturing and reducing conditions on 4-12% NuPAGE gels (Invitrogen) and transferred to polyvinylidene fluoride membranes. Immunoreactive proteins were revealed by enhanced chemiluminescence with ECL (Perkin-Elmer). An antibody against phosphorylated CamKII (Cell Signaling, catalog number: 3361) was used.

Nuclear Volume Evaluation

Nuclei were stained with DAPI and nuclear volume was evaluated with ImageJ software.

Centrosome Labelling

Cells were grown on glass coverslips for 24 h to allow cultures to reach 80% confluence. To measure the number of centrosomes, cells were fixed with 4% PFA, permeabilized with methanol at −20° C. for 8 min and blocked with 0.5% Triton X-100 in PBS for 30 min at RT. To detect g-tubulin, cells were incubated overnight at 4° C. with a mouse anti-g-tubulin antibody (GTU-88; Sigma) diluted 1/1000 in PBS containing 0.1% milk and 0.05% Triton X-100. After washing, the cells were incubated for 1 h at RT with Cy3-conjugated goat antimouse IgG secondary antibody (Caltag Laboratories) diluted 1/2000 in PBS containing 0.1% milk and 0.05% Triton X-100. The preparations were counterstained with DAPI in Vectashield mounting medium (Vector Laboratories). Fluorescent images were acquired with a microscope (Leica DMR) equipped with a PL APO objective.

Connexon Activity

One hundred thousand cells were cultured as described above for one day with or without CamKII inhibitors (KN62 or KN93 at a final concentration of 10 μM). Lucifer yellow (LY) was added to the medium (final concentration: 110 mM) and incubated for two hours. Fluorescence was recorded using a Perkin Elmer Victor 4 microplaque reader (excitation: 405 nM, emission: 535 nM).

Animal Treatment

Transgenic animals received intraperitoneal injections, either with a placebo (0.9% physiological serum) or with KN 93 (1.5 mg/kg). Treatment took place every day for one month.

Locomotor Test

Locomotor ability was tested using the rotarod procedure, requiring motor coordination and balance control. A machine manufactured by Bioseb was used for this purpose. A previously used procedure (Norreel et al., 2001) was adapted and two velocities (15 and 25) were used. Briefly, each mouse underwent the same 5-day procedure. The first two days were used to train the animals (five sessions of 2-min walking at a very low speed, i.e. five rotations per minute (r.p.m.)). The last three days were used to run the test sessions. Each day, the mice performed two series of five trials, a training speed with a 1-h rest period between the two series and a 5-min rest period between two consecutive trials. The software provided by Bioseb (Sedacom v1) was used to monitor the time the animals were on the rod.

Statistical Analysis

Statistical analysis was performed using Prism v5.0. Mann-Whitney and chi-square tests were used for trend analysis. The significance threshold was p<0.05.

Results:

Centrosomes Over Duplication. Mechanism and Correction.

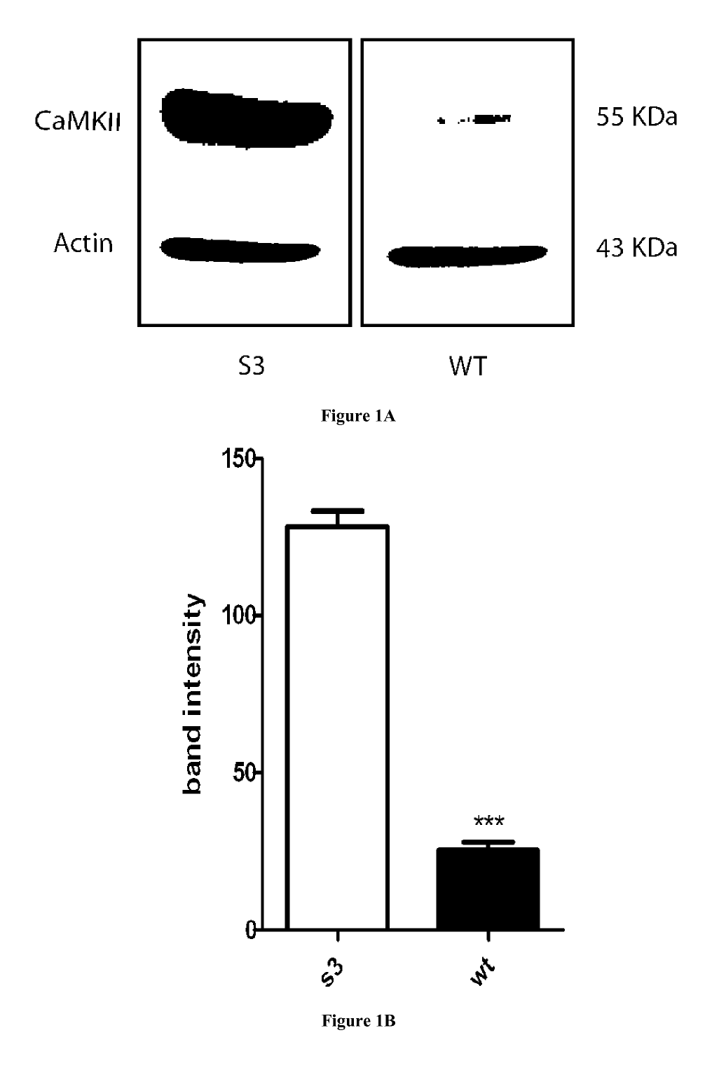

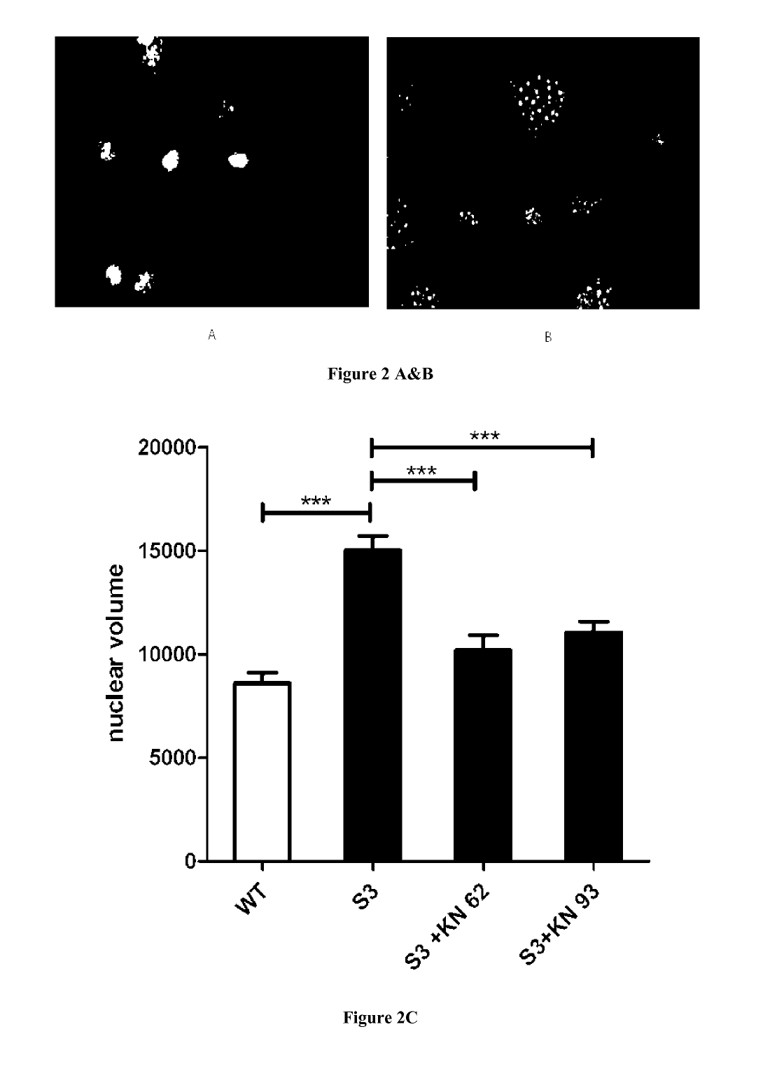

Centrosome over-duplication has been linked to over-activation of CamKII (Ca2+/calmodulin-dependent protein kinase II) (Matsumoto and Maller, 2002). Therefore, CamKII activity was evaluated in fibroblasts from normal or transgenic lines. FIGS. 1A and B shows that the phosphorylated form of CamKII is strongly increased in S3 fibroblasts, indicating enzymatic over-activity in transgenic lines, which could explain the centrosome over-duplication observed in transgenic lines (FIGS. 2A and B). In order to corroborate these results and to explore a potential rescue of the cellular phenotype (centrosome overduplication), fibroblastic cell lines derived from transgenic animals were incubated with two reference CamKII inhibitors, KN93 and KN62, which have different chemical structures (Tokumitsu et al., 1990; Sumi et al., 1991). In addition, KN92, a very close analogue of KN93 but presenting no CamKII inhibitory activity, was used as a negative control. In the absence of CamKII inhibitor, all transgenic fibroblasts present aneuploidy, which could be detected by evaluation of the nuclear volume. FIG. 2C and table 1 shows that treatment with either KN62 or KN93 resulted in restitution of normal cellular volume. In the same S3 line, we evaluated the percentage of cells presenting one, two or more than two centrosomes. FIG. 2D (and Table 2) shows that centrosome number per cell returns to close to normal after treatment with either KN93 or KN62. In contrast, abnormal centrosome number per cell is unchanged in cells treated with KN92, an analogue of KN93 that does not inhibit CamKII (table 2). These results strongly suggest that involvement of Cx32 in chromosome stability is mediated through centrosome duplication, involving the CamKII signalling pathway.

Connexon Activity.

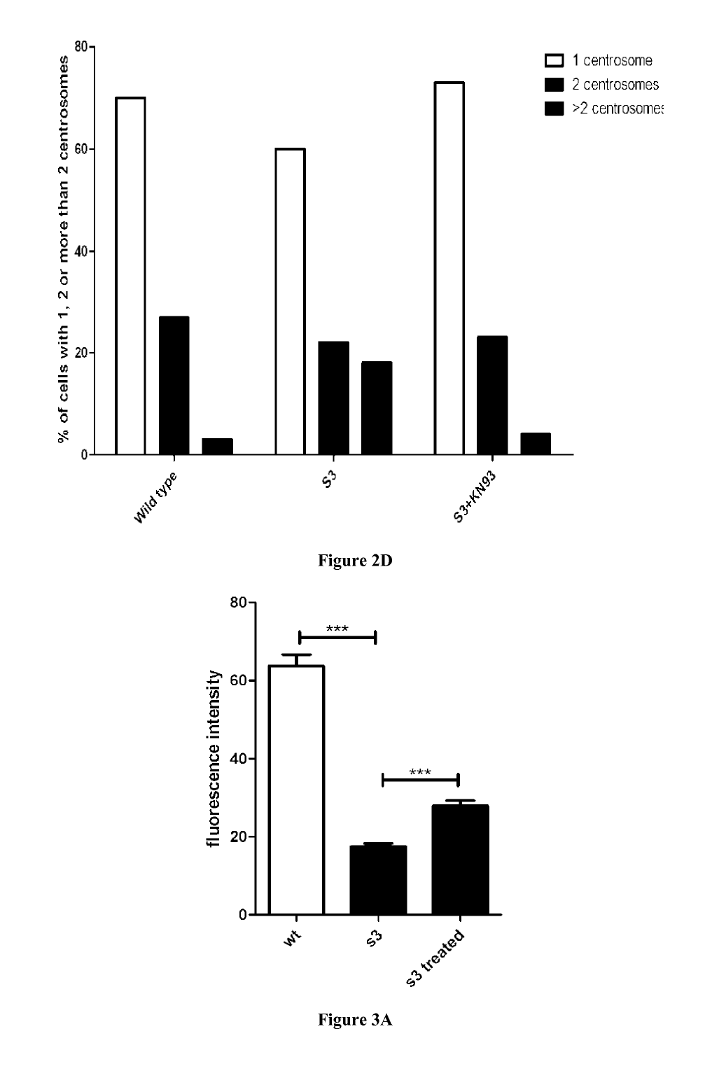

However, these results did not allow us to conclude that CamKII inhibitors can rescue the connexon activity caused by Cx32 mutations. We therefore analyzed connexon activity in WT and S3 fibroblasts, treated or not with KN93. For this purpose we used lucifer yellow dye, which becomes fluorescent when it enters cells. Moreover, internalisation of this dye specifically indicates connexon activity. We observed in FIG. 3 A, B, C, D (and in table 3) that connexon activity is severely reduced in S3 cells, and that the connexon activity is significantly improved though not totally restored by KN93 treatment. These data demonstrate that mitotic instability, CamKII activity and connexon failure are linked. In addition, we demonstrated that the CamKII inhibitor KN93 could be a potential drug for treating the CMTX phenotype.

Impact of CamKII Inhibitors on Locomotor Performances.

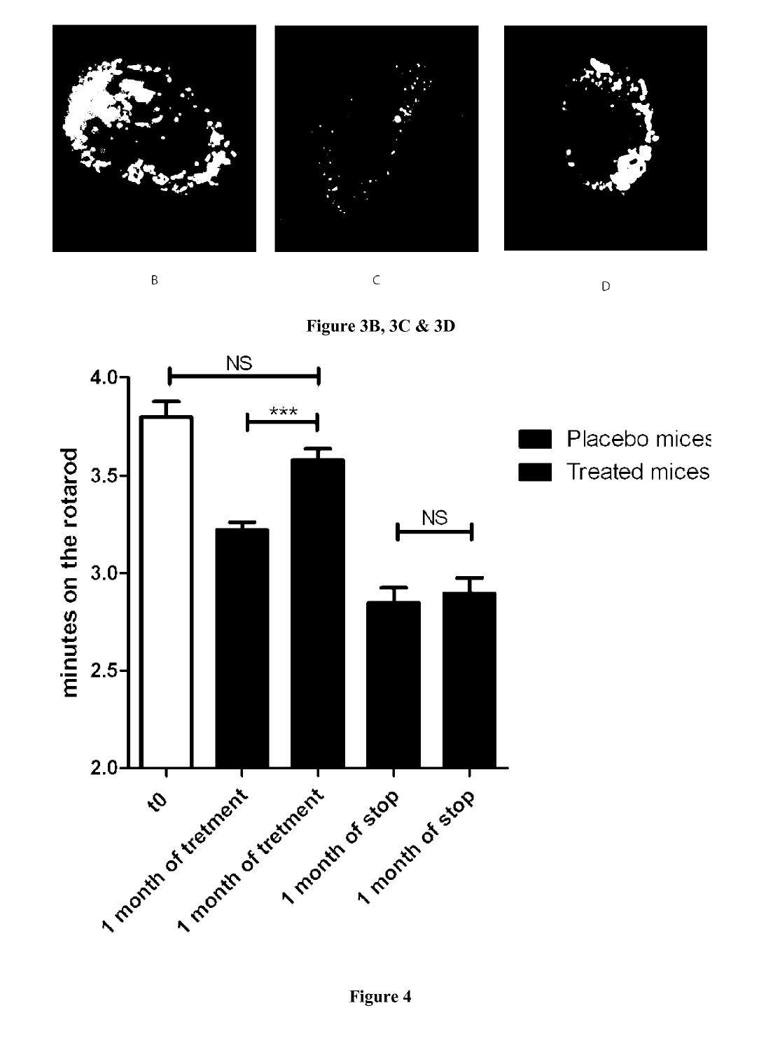

In a further step, we analyzed the impact of KN93 treatment on locomotor impairment in our transgenic models. The S3 and G2 transgenic mouse lines were treated with a soluble form of KN93 (1.5 mg/kg i.p. per day). These two lines present locomotor impairment, appearing at about seven months of age and progressing with age (Mones et al., 2012). FIG. 4 (and table 4) shows that treatment of the G2 animals with KN93 significantly lowers the progression of the phenotype. In contrast, placebo-treated animals still presented progressive locomotor degradation of the phenotype with age. In addition, when KN93 treatment was stopped after one month, rapid degradation of the locomotor activity ensued. In addition, S3 animals were treated with KN93 and the degradation of locomotor capacities in this transgenic line was lowered as for the G2 line (table 5). As previously observed, stopping the treatment led to a strong degradation of locomotion.

Discussion

We recently demonstrated (Mones et al., 2012) that connexin 32, involved in the Xlinked form of Charcot-Marie-Tooth disease, is involved in mitotic stability. We suggested here that this instability presented by mutated proteins, is due to CamKII over expression, leading to centrosome over duplication. Matsumoto and Maller (2002) have demonstrated the relation between calcium flux, CamKII activity and centrosome duplication. In 1997, Torok et al. identified two calmodulin-binding amino-acid sequences in connexin 32, and provide evidences that calmodulin may function as an intracellular ligand, regulating Ca2+ dependent intercellular communication across gap junctions. An examination of these specific calmodulin binding sites identified two regions, one at the N-terminus, and another at the C-terminus, showing calcium-independent calmodulin-binding properties (Ahmad et al., 2001). Using a truncation mutant Cx32D215 they demonstrate that aminoacid sequences in the third transmembrane domain and a calmodulin-binding domain in the cytoplasmic tail of Cx32 are likely candidates for regulating connexin oligomerization. Finally Dodd et al., (2002) demonstrate that this physical proximity of Cx32 and CamKII has a physiological role. It is thus likely that pathological mutations in Cx32, associated to CMTX, results in mitotic instability through CamKII over expression leading to centrosome overduplication. It has been observed by us (Mones et al., 2012) and by the European Mitocheck project (www.mitocheck.org) that a lowering of Cx32 expression or expression of a mutated isoform, resulted in perturbation in cell division. Control of cell division is a key event in Schwann cell division. Moreover perturbation of cell division has been associated to defect in myelination in experimental models as well as in patients nerves (for review, Muller at al, 2000). This is likely the basis of anomalies in myelination observed in CMTX. Treatment with CamKII inhibitors (KN62 or KN93) resulted in a partial rescue of the cellular phenotype (abnormal centrosome over-duplication) and in a partial restoration of connexon activity. In addition, in vivo KN93 treatment of CMTX-related transgenic mice, although not fully correcting the degenerative phenotype, significantly lowered the degradation of locomotor performance. These data confirm that CamKII are a downstream target of Cx32 activity and that CamKII inhibitors could be thus the first therapeutic class of molecules with potential for use in treating CMTX, a rare disorder for which no curative treatment is known. Moreover, our findings suggested that CamKII inhibitors could be a good target to cure disorder in which mitotic instability has also been observed as polykystic kidney disease (Battini et al., 2008; Burtey et al., 2008).

TABLE 1

| | mouse lines | | | |
|---|---|---|---|---|
| | WT | S3 | S3 + KN62 | S3 + KN93 |
| Nuclear | 8607 +/− | 15029 +/− | 10100 +/− | 11000 +/− |
| Volume | 508 | 690 | 729 | 540 |

Nuclear volume correction. Fibroblasts from Wild type (WT) and S3 transgenic mouse have been cultured for 24 hrs with or without CamKII inhibitors (KN62 and KN93). Nuclear volume has been evaluated using the Imagej software. Data have been analyzed using the Mann-Whitney test, demonstrating that differences are not statistically different between treated cells and wild type and highly statistically significant between Non treated and treated cells (pValue<0.0001). This experiment has been repeated three time.

TABLE 2

| Mouse lines | WT | S3 | S3 + KN 62 | S3 + KN 93 | S3 + KN92 |
|---|---|---|---|---|---|
| 1 centro | 79% | 60% | 62% | 73% | 60% |
| 2 centro | 18 | 22 | 32 | 20 | 20 |
| >centro | 3 | 18 | 6 | 7 | 20 |

Number of centrosomes per cell. Fibroblasts from WT and S3 have been cultured with or without CamKII inhibitors. Centrosome number per cell has been evaluated as described in methods. Results have been analyzed using the Chi2 trends for tendency test demonstrating that data distribution is highly significant between treated and non treated cells (p value<0.0001). This experiment has been repeated three time.

TABLE 3

| Mouse lines | WT | S3 | S3 + KN93 |
|---|---|---|---|
| LY intensity | 63 +/− 17 | 17 +/− 5 | 28 +/− 9 |

Fibroblasts from WT and S3 (treated or not with KN93) have been cultured for 2 days in 96 wells microplaques. Lucifer yellow has been then added to the medium and fluorescence monitored after 2 hours (cell auto fluorescence and fluorescence of LY without cells, have been evaluated and substracted). This experiment has been repeated three time.

TABLE 4

| | | | | W4 | W4 |
|---|---|---|---|---|---|
| | | | | placebo + | treated + |
| | | | | 1 month | 1 month |
| G2 line | T0 | W4 placebo | W4 treated | arrest | arrest |
| Mean | 3.90 | 3.20 | 3.6 | 2.84 | 2.9 |
| Minutes on | | | | | |
| rotarod | | | | | |
| St error | 0.2 | 0.15 | 0.22 | 0.28 | 0.28 |

Mouse from the G2 transgenic line have been treated either with a placebo (4 animals) or with soluble KN93 (4 animals). Locomotor performances of the animals have been evaluated using the rotarod test after one month of treatment. Performances of WT and G2 treated with KN93, are not statistically different. On the contrary performances of G2 animals treated either with a placebo or with KN93 are highly statistically significant (p value<0.0001). Treatment has been stopped for one month and rotarod performances evaluated. Rotarod performances of KN 93 treated or placebo treated degrade and are not statically different.

SUPPLEMENTARY TABLE 5

| | | | | W4 | W4 |
|---|---|---|---|---|---|
| | | | | placebo + | treated + |
| | | | W4 | 1 month | 1 month |
| S3 line | T0 | W4 placebo | treated | arrest | arrest |
| Mean Minutes | 2.7 | 2.1 | 2.5 | 1.5 | 1.41 |
| on rotarod | | | | | |
| St error | 0.28 | 0.13 | 0.11 | 0.23 | 0.29 |

Mouse (4) from the S3 transgenic line have been treated either with a placebo or with soluble KN93. Locomotor performances of the animals have been evaluated using the rotarod test after one month of treatment. Performances of WT and S3 treated with KN93, are not statistically different. On the contrary performances of S3 animals treated either with a placebo or with KN93 are highly statistically significant (p value<0.0001). Treatment has been stopped for one month and rotarod performances evaluated. Rotarod performances of KN 93 treated or placebo treated degrade and are not statistically different.

### EXAMPLE 14

### Biological Effects of KN62 and KN93 Derivatives

Analogues KN62 and KN93 molecules, which we have shown that they corrected the phenotype of a mouse model of the disease CMTX were synthesized according to EXAMPLES 1-12.

Animal cells and wild-type mouse cells transgenic animals S3 created in the as described in EXAMPLE 13 are cultured in 96-well microplates. We have shown that the S3 cells have an abnormality of the connexon activity due to the mutation of connexin 32 introduced in this line. Moreover, the cells exhibited mitotic instability with polyploidy (Mones et al, Glia, 2012). The cells were incubated with new molecules and connexon activity was evaluated. We show that all substitutions of side chains in the molecule KN62 suppress the activity of the molecule. Regarding KN93 derivatives most substitutions suppress the activity “correction activity connexon” molecules, excepting for a few molecules. The results are shown in Table A. We can see that only molecule CM-08-33 shows an activity which is similar to KN93 at a concentration 10 times lower (1 μM).

TABLE A

| | WT | S3-trait | KN93 | 0.1 μM | 1 μM | 10 μM | 50 μM |
|---|---|---|---|---|---|---|---|
| CM08-16 | 230 | 126 | 191 | 131 | 142 | 159 | 160 |
| CM08-18 | 230 | 129 | 191 | 125 | 150 | 162 | 174 |
| CM08-33 | 230 | 129 | 191 | 162 | 196 | 189 | 180 |
| CM08-40 | 230 | 126 | 191 | 169 | 169 | 167 | 162 |

S3 cells were treated with various molecules, at increasing concentrations. Moreover transgenic animals of the same lineage cells and untreated transgenic animals are used as controls. The connexon activity was assessed after 24 hours of treatment. The activity is expressed in thousands of arbitrary fluorescence units. Treatment with concentrations of 0.1 mM, is not significant for 16 and 18 but is 33 and 40 (pvalue 0.0013 and 0.0048). As a comparison we use the reference molecule KN 93 at a concentration of 10 μM.

### REFERENCES

Throughout this application, various references describe the state of the art to which this invention pertains. The disclosures of these references are hereby incorporated by reference into the present disclosure.
Abrams, C. K., Freidin, M. M., Verselis, V. K., Bennett, M. V. and Bargiello, T. A. 2001.
Functional alterations in gap junction channels formed by mutant forms of connexin 32: evidence for loss of function as a pathogenic mechanism in the X-linked form of Charcot-Marie-Tooth disease. Brain Res. 900: 9-25.
Ahmad, S., Martin, P. E. and Evans, W. H. 2001. Assembly of gap junction channels: mechanism, effects of calmodulin antagonists and identification of connexin oligomerization determinants. Eur J Biochem. 268(16):4544-4552
Battini, L., Macip, S., Fedorova, E., Dikman, S., Somlo, S., Montagna, C. and Gusella, G. L. 2008. Loss of polycystin-1 causes centrosome amplification and genomic instability. Hum Mol Genet. 17(18):2819-2833.
Bennett, M. V., Barrio, L. C., Bargiello, T. A., Spray, D. C., Hertzberg, E. and Sáez, J. C. 1991. Gap junctions: new tools, new answers, new questions. Neuron 6 (3):305-320.
Bergoffen, J., Scherer, S. S., Wang, S., Scott, M. O., Bone, L. J. 1993. Connexin mutations in X-linked Charcot-Marie-Tooth disease. Science 262:2039-2042.
Bicego, M., Morassutto, S., Hernandez, V. H., Morgutti, M., Mammano, F. 2006. Selective defects in channel permeability associated with Cx32 mutations causing X-linked Charcot-Marie-Tooth disease. Neurobiol. Dis. 21 (3):607-617.
Boerkel, C. F., Takashima, H., Garcia, C. A., Olney, R. K., Johnson, J., Berry, K. 2002. Charcot-Marie-Tooth disease and related neuropathies: mutation distribution and genotypephenotype correlation. Ann. Neurol. 51:190-201.
Burtey, S., Riera, M., Ribe, E., Pennenkamp, P., Rance, R., Luciani, J., Dworniczak, B., Mattei, M. G. and Fontés, M. 2008. Centrosome overduplication and mitotic instability in PKD2 transgenic lines. Cell Biol Int. 32(10):1193-1198.
De Jonghe, P., Nelis, E., Timmerman, V., Löfgren, A., Martin, J. J. and Van Broeckhoven, C, 1999. Molecular diagnostic testing in Charcot-Marie-Tooth disease and related disorders. Approaches and results. Ann. N. Y. Acad. Sci. 883:389-396.
Deschênes, S. M., Walcott, J. L., Wexler, T. L., Scherer, S. S. and Fischbeck, K. H. 1997. Altered trafficking of mutant connexin32. J. Neurosci. 17 (23); 9077-9084.
Dodd, R., Peracchia, C., Stolady, D. and Torok, K. 2008. Calmodulin association with connexin32-derived peptides suggests trans-domain interaction in chemical gating of gap junction channels. J Biol Chem. 283(40):26911-22690.
Dyck, P. J. and Lambert, E. H. 1968. Lower motor and primary sensory neuron diseases with peroneal muscular atrophy. I. Neurologic, genetic, and electrophysiologic findings in hereditary polyneuropathies. Arch. Neurol. 18 (6):603-618.
Hahn, A. F., Brown, W. F., Koopman, W. J. and Feasby, T. E. 1990. X-linked dominant hereditary motor and sensory neuropathy. Brain 113:1511-1525.
Huxley, C., Passage, E., Manson, A., Putzu, G., Figarella-Branger, D., Pellissier, J. F. and Fontes, M. 1996. Construction of a mouse model of Charcot-Marie-Tooth disease type 1A by pronuclear injection of human YAC DNA. Human Molecular Genetics 5:563-569.
Kumar, N. M. and Gilula, N. B. 1992. Molecular biology and genetics of gap junction channels. Semin. Cell. Biol. 3 (1):3-16.
Liu, F., Arce, F. T., Ramachandran, S. and Lal, R. 2006. Nanomechanics of hemichannel conformations: connexin flexibility underlying channel opening and closing. J. Biol. Chem. 281 (32):23207-23217.
Matsumoto, Y., and Maller, J. L. 2002. Calcium, calmodulin, and CaMKII requirement for initiation of centrosome duplication in Xenopus egg extracts. Science 295:499-502.
Mese, G., Richard, G., and White, T. W. 2007. Gap junctions: basic structure and function. J. Invest. Dermatol. 127 (11):2516-2524.
Mones, S., Bordignon, B. and Fontes, M. 2012. Connexin 32 is involved in mitosis. Glia 60 (3):457-64.
Müller, H. W. 2000. Tetraspan myelin protein PMP22 and demyelinating peripheral neuropathies: new facts and hypotheses. Glia 29(2):182-185.
Oh, S., Ri, Y., Bennett, M. V., Trexler, E. B., Verselis, V. K. and Bargiello, T. A.1997. Changes in permeability caused by connexin 32 mutations underlie X-linked Charcot-Marie-Tooth disease. Neuron 19 (4):927-938.
Norreel, J. C., Jamon, M., Riviere, G., Passage, E., Fontés, M. and Clarac, F. 2001. Behavioural profiling of a murine Charcot-Marie-Tooth type 1A model. Eur. J. Neurosci. 8:1625-1634.
Omori, Y., Mesnil, M. and Yamasaki, H. 1996. Connexin 32 mutations from X-linked Charcot-Marie-Tooth disease patients: functional defects and dominant negative effects. Mol. Biol. Cell. 7:907-916.
Scherer, S. S., Deschenes, S. M., Xu, Y. T., Grinspan, J. B., Fischbeck, K. H. and Paul, D. L. 1995. Connexin32 is a myelin-related protein in the PNS and CNS. J. Neurosci. 15 (12):8281-8194.
Sumi, M., Kiuchi, K., Ishikawa, T., Ishii, A., Hagiwara, M.1991. The newly synthesized selective Ca2+/calmodulin dependent protein kinase II inhibitor KN-93 reduces dopamine contents in PC12h cells. Biochem. Biophys. Res. Commun. 181(3):968-75.
Tokumitsu, H., Chijiwa, T., Hagiwara, M., Mizutani, A., Terasawa, M. 1990. KN-62, 1-[N,Obis(5-isoquinolinesulfonyl)-N-methyl-L-tyrosyl]-4-phenylpiperazine, a specific inhibitor of Ca2+/calmodulin-dependent protein kinase II. J. Biol. Chem. 265 (8):4315-20.
Torok, K, Stauffer, K. and Evans, W. H. 1997. Connexin 32 of gap junctions contains two cytoplasmic calmodulin-binding domains. Biochem. J. 326:479-483
VanSlyke, J. K., Deschenes, S. M. and Musil, L. S. 2000. Intracellular transport, assembly, and degradation of wild-type and disease-linked mutant gap junction proteins. Mol. Biol. Cell. 11:1933-1946.
Wang, H. L., Chang, W. T., Yeh, T. H., Wu, T., Chen, M. S. and Wu, C. Y. 2004. Functional analysis of connexin-32 mutants associated with X-linked dominant Charcot-Marie-Tooth disease. Neurobiol. Dis. 15 (2):361-370.
Yoshimura, T., Ohnishi, A., Yamamoto, T., Fukushima, Y., Kitani, M. and Kobayashi, T. 1996. Two novel mutations (C53S, S26L) in the connexin32 of Charcot-Marie-Tooth disease type X families. Hum. Mutat. 8 (3):270-272.
Yum, S. W., Kleopa, K. A., Shumas, S. and Scherer, S. S. 2002. Diverse trafficking abnormalities of connexin32 mutants causing CMTX. Neurobiol. Dis. 11 (1):43-52.

1. A method for the treatment of CMTX in a subject in need thereof comprising administering to the subject a therapeutically effective amount of an inhibitor of CamKII activity or expression.

2. The method according to claim 1 wherein the inhibitor of CamKII activity is selected from the group consisting of KN-93, KN-62, N′-{3-[1-(2-fluorobiphenyl-4-yl)-ethyl]-isoxazol5-yl}-N,N-dimethyl-guanidine hydrochloride, N-ethyl-N′-{3-[1-(2-fluorobiphenyl-4-yl)-ethyl]isoxazol-5-yl}-guanidine hydrochloride; 4-[1-(2-fluorobiphenyl-4-yl)ethyl]-2-(methylamino)-ethylthiazole, 1[1-oxo-11-(5,6-dihydro-3-iminobenzo[h]cinnolin-2(3H)-yl)undecyl]-4-(2-pyrimidinyl)-piperazine, 2-hydroxy-S-(4-methoxyphenyl)-N′-[2-({methyl[(2E)-3-phenylprop-2-en-1-yl]amino}methyl)phenyl]ethane-1-sulfonamido, 2-hydroxy-S-(4-methoxyphenyl)-N-(2-{[methyl(naphthalen-2-ylmethyl)amino]methyl}phenyl)ethane-1-sulfonamido, N-(2-{[benzyl(methyl)amino]methyl}phenyl)-2-hydroxy-S′-(4-methoxyphenyl)ethane-1-sulfonamido, and N-[2-({[3-(4-chlorophenyl)propyl](methyl)amino}methyl)phenyl]-2-hydroxy-S′-(4-methoxyphenyl)ethane-1-sulfonamido.

3. The method of claim 1 wherein the inhibitor of CamKII activity is a peptide having amino acid sequence Lys-Lys-X-Leu-Arg-Arg-Gln-Glu-Ala-Phe-Asp-Ala-Tyr (SEQ ID NO:1) wherein X is Ala or Lys or a peptide having amino acid sequence Lys-Lys-Ala-Leu-His-Arg-Gln-Glu-Ala-Val-Asp-Cys-Leu (SEQ ID NO:2).

4. A compound selected from the group consisting of 2-hydroxy-S-(4-methoxyphenyl)-N′-[2-({methyl[(2E)-3-phenylprop-2-en-1-yl]amino}methyl)phenyl]ethane-1-sulfonamido; 2-hydroxy-S-(4-methoxyphenyl)-N′-(2-{[methyl(naphthalen-2-ylmethyl)amino]methyl}phenyl)ethane-1-sulfonamido; N-(2-sulfonamido; and N-[2-({[3-(4-chlorophenyl)propyl](methyl)amino}methyl)phenyl]-2-hydroxy-S′-(4-methoxyphenyl)ethane-1-sulfonamido.
